(19) United States
(12) Patent Application Publication
(10) Pub. No.: US 2002/0025508 A1
(43) Pub. Date: Feb. 28, 2002

(54) Process for finding a protease inhibitor

(76) Inventors: Katja Fechteler, Wiesbaden (DE); Marcus Kostka, Mainz (DE); Klaus Fuchs, Gau-Algesheim (DE); Christian Haass, Icking (DE); Harald Steiner, Muenchen (DE)

Correspondence Address: BOEHRINGER INGELHEIM CORPORATION, 900 RIDGEBURY ROAD, P O BOX 368, RIDGEFIELD, CT, 06877, US

(21) Appl. No.: 09/755,417

(22) Filed: Jan. 5, 2001

Related U.S. Application Data
(60) Provisional application No. 60/177,145, filed on Jan. 20, 2000.

(30) Foreign Application Priority Data
Jan. 6, 2000 (DE) 100 00 161.0.

(51) Int. Cl. C12Q 1/00; A61K 31/00

(52) U.S. Cl. 435/4; 514/1

(57) ABSTRACT

The present invention relates to a process for finding substances capable of specifically inhibiting membrane-bound proteases and a high throughput screening test for finding substances capable of specifically inhibiting γ-secretase or presenilinase. The invention further relates to the use of this process or this high throughput screening test for finding substances which are specifically capable of inhibiting γ-secretase or presenilinase. It also discloses substances which can be found with a process according to the invention, the use of said substances according to the invention for preparing a medicament for treating neurodegenerative disorders, particularly Alzheimer's disease, and pharmaceutical formulations containing the substances according to the invention.

### RELATED APPLICATIONS

The benefit of prior provisional application Ser. No. 60/177,145, filed Jan. 20, 2000 is hereby claimed.

### BACKGROUND

The present invention is in the field of processes for finding inhibitors of membrane-based proteases; these are in particular γ-secretase and presenilinase inhibitors. Another embodiment of the invention relates to the use of these inhibitors for preparing a medicament for the treatment of neurodegenerative diseases as well as pharmaceutical formulations which contain these substances. The aggregation and precipitation of proteins are involved in the origins of various neurodegenerative diseases such as Alzheimer's, Parkinson's and Huntington's chorea. In Alzheimer's disease the amyloid-β-peptide (Aβ) aggregates and leads to insoluble senile plaques which constitute one of the pathological markers of the disease (Selkoe, D. J. et al., J. Biol. Chem., 1996, 271:18295-18298). Aβ is formed by the proteolytic cleaving of a precursor protein, the amyloid precursor protein (APP). Two metabolic routes of APP have been distinguished, the non-amyloidogenic route and the amyloidogenic route (Selkoe, D. J., Annu. Rev. Cell Biol., 1994, 10:373-403). In the non-amyloidogenic metabolism of APP, α-secretase cleaves within the Aβ region of the APP and thus leads to the secretion of the soluble N-terminal region of the protein (α-APP) and after the cutting of γ-secretase leads to the release of p3. By contrast, the amyloidogenic route leads to the formation of Aβ, as two proteases generate the N-terminus (β-secretase) or C-terminus (γ-secretase) of Aβ (Haass,C. and Selkoe, D. J., Cell, 1993, 75:1039-1042; Selkoe, D. J. , Annu. Rev. Cell Biol., 1994, 10:373-403).

In vivo, Aβ can be detected in human plasma and cerebrospinal fluid. Secreted Aβ can also be detected in cell culture, in the cell culture supernatant of various cell types which endogenously express or overexpress APP or fragments thereof. Both Aβ production and hence the formation of amyloid plaques are influenced by various genetic risk factors. These include mutations in the homologous proteins presenilin 1 and presenilin 2 and in APP itself. The analysis of these mutations on fibroblasts from Alzheimer's patients with familiar Alzheimer's disease (FAD) demonstrated the influence which they have on Aβ production. This was confirmed by investigations on transfected cells and transgenic animals. All mutations increase the production of Aβ and, in the case of the presenilin mutations, lead to a selective increase in the longer Aβ variant, Aβ42 (Selkoe, D. J., J. Biol. Chem., 1996, 271:18295-18298; Price, D. L. et al., Ann. Rev. Genet., 1998, 32:461-493). This peptide aggregates to a greater extent than the shorter form, Aβ40, and has a more toxic effect on cells of neuronal origin than on those of peripheral origin (Lemere, C. A. et al., Nat. Med., 1996, 2:1146-1150; Mann, D. M. et al., Ann. Neurol., 1996, 40:149-156). In addition to this effect of the mutations, there are indications that the wild type form of the presenilins also have a fundamental function in the physiological formation of Aβ. In neurons from mouse embryos in which the PS1-gene (PS: presenilin) had been switched off by genetic engineering, there was found to be a drastic reduction in Aβ40 and Aβ42. Moreover, the C-terminal fragments of APP accumulated in the cells, leading to the view that the presenilins activate γ-secretase or themselves have γ-secretase activity (De Strooper, B. et al., Nature, 1998, 391:387-390; Sisodoa, S. et al., 1998, Ann. Rev. Neuroscience, 21:479-505). Early in vitro test systems combined with mutation studies on conserved aspartates of presenilin 1 led one to assume that the presenilins could be special autocatalytically active aspartate proteases which are responsible for the γ-secretase in the membrane (Wolfe, M. S. et al., Nature, 1999 398:513-517).

The discussion of the identification of γ-secretase as the decisive step in the generation of Aβ and hence in the origins of Alzheimer's has still not concluded. Quite independently, this protease constitutes an interesting target for intervening pharmacologically in the process of Aβ formation by finding inhibitors which selectively reduce the activity thereof. It is important to develop not only animal models and in vitro test systems but also cellular assays which will make it possible to test specific active substances irrespective of transporting processes within the cell.

Wolfe, M. S. et al., Nature, 1999, 398:513-517 have disclosed an in vitro system for measuring the activity of γ-secretase. In order to prepare the system, membranes from cells which express PS1 in a stable manner are worked up. They are mixed with a plasmid coding for the LC99-polypeptide and subjected to an in vitro transcription/translation reaction in the presence of ³⁵S-methionine to form the γ-secretase substrate C99. The mixture is then incubated under the appropriate conditions, during which time the C99 fragment is proteolytically cleaved from APP by the γ-secretase, and the breakdown products are detected by gel electrophoresis after immunoprecipitation. This test system is very time-consuming and incapable of being automated and is not therefore suitable for finding specific protease inhibitors.

At the moment, there are no known therapeutic approaches for preventing neuronal cell death on the basis of presenilin fragments which appear to be involved in neuronal apoptosis. Presenilinase, i.e. the protease activity which generates said presenilin fragments, is an excellent target molecule. No methods are known from the prior art for finding presenilinase inhibitors, particularly not in an industrial context.

The aim of the invention is to provide an improved test system for finding specific protease inhibitors. This aim can be achieved by means of the present invention within the scope of the specification and claims.

Before the present invention is described in more detail, it should be pointed out that all the plurals also include the singulars and vice versa, i.e., if a reference is made to “the substances” individual substances are also covered by the invention.

### SUMMARY OF THE INVENTION

The present invention relates to a process for finding substances capable of specifically inhibiting membrane-based proteases, and a high throughput sampling test for finding substances which are capable of specifically inhibiting γ-secretase or presenilinase. The invention further relates to the use of this process or this high throughput sampling test for finding substances which are able to specifically inhibit γ-secretase or presenilinase. The invention also discloses substances which can be found with a process according to the invention, the use of said substances according to the invention for preparing a medicament for treating neurodegenerative diseases, particularly Alzheimer's disease, and pharmaceutical formulations which contain the substances according to the invention.

### SUMMARY OF THE FIGURES

FIG. 1: Preparation of reporter constructs and cell lines.

FIG. 2: Carrying out the test system—HTS assay principle.

FIG. 3: ER retention of the γ-secretase substrate—intracellular APP processing.

FIGS. 4A-4D: Characterisation of the transfected cell lines for HTS.

A) Determining the Aβ-KKK/Gal4 substrate expression by Western blot.

B) Determining the luciferase expression by Western blot.

C) Determining the luciferase expression by enzymatic detection.

D) Determining the luciferase expression by enzymatic detection.

FIG. 5: Determining the levels of extracellular Aβ42 secreted by the transfected HTS test cell lines.

FIGS. 6A-6B: BFA (Brefeldin A) effect in the HTS test cell lines Aβ-KKK-ER/59 and Aβ-KKK/52.

A) Luciferase test.

B) Vitality test.

FIGS. 7A-7C: Effect of presenilin (PS1 and PS2) aspartate mutations in the HTS test cell lines Aβ-KKK-ER/59 and Aβ-KKK/52

A) Effect of the PS (Asp) mutants in the pool cells Aβ-KKK/52/DN385N (PS1mut) and Aβ-KKK/52/D366A (PS2mut).

B) Effect of the PS (Asp) mutants in the pool cells Aβ-KKK-ER/59/D385N (PS1mut) and Aβ-KKK-ER/59/D366A (PS2mut).

C) Detection of overexpression of the PS (Asp) mutants in the particular cell lines.

FIGS. 8A-8B: Substance A.

A) Concentration-dependent inhibition of Aβ formation by substance A.

B) Concentration-dependent inhibition of Aβ 40 and Aβ 42 secretion by substance A.

FIG. 9: Substance B.

Concentration dependent inhibition of Aβ formation by substance B.

FIG. 10: Substance C.

Concentration dependent inhibition of Aβ formation by substance C.

### DETAILED DESCRIPTION OF THE INVENTION

According to the invention, a process is provided for finding substances capable of specifically inhibiting proteases which cleave membrane-based substrates which is characterised in that:

a) cells are cultivated which have said protease activity endogenously or exogenously and express a membrane-associated recombinant fusion protein which comprises the substrate of said protease with the specific cleavage site for said protease and a reporter;

b) these cells are incubated with a test substance;

c) the quantity of reporter cleaved is measured; and

d) the value obtained is compared with the value obtained in the absence of the test substance.

By said protease or protease activity is meant an enzyme or an enzymatic activity which cleaves specifically, i.e., between certain amino acid sequences, and does not cleave between other amino acid sequences, and the substrates of which are membrane-based or are membrane-associated. By membrane-based is meant, within the scope of this invention, that the substrate is an integral part of the membrane. By membrane associated, for the purposes of the invention, is meant that the substrate is bound to the surface of the membrane or to integral membrane proteins. This definition of membrane associated substrates should also include substrates which interact via chemical groupings with the hydrophobic part of the membrane which are added by posttranslational modifications. Moreover, the term membrane associated substrates should also include substrates which interact via amino acid side chains with the hydrophobic part of the membrane, albeit to a lesser extent than the integral membrane proteins. As an example, prostaglandin synthetase may be mentioned here. By substrate is meant peptide and proteins which contain at least one cleavage site of said protease. The substrates may also be modified, i.e., glycosylated, for example (Sambrook et al., 1989, Molecular Cloning: A Laboratory Manual, Cold Spring Harbor Laboratory, Cold Spring Harbor, N.Y.). According to the invention, said substrate is fused to a reporter (see below) which serves to detect the protease activity (cleaving). The fusion proteins according to the invention may be produced by current methods of genetic engineering (Sambrook et al., 1989, Molecular Cloning: A Laboratory Manual, Cold Spring Harbor Laboratory, Cold Spring Harbor, N.Y.) if the DNA coding for the substrate and reporter protein is available. The DNA which codes for the reporter proteins may be obtained, for example, from commercial suppliers such as Clontech of Heidelberg and inserted into the desired vectors by standard procedures (Sambrook and Maniatis, Molecular Cloning: A Laboratory Manual, Cold Spring Harbor Laboratory, Cold Spring Harbor, N.Y., 1989). The DNA which codes for the substrate can be obtained for proteases from suitable gene banks using standard methods.

Test substances may be any substances known in the art or new substances. For example, these substances may be proteins or chemical compounds which are also available in commercial libraries of substances. By comparing the value obtained with the value obtained in the absence of the test substance, as described in part d) of the process according to the invention, the specificity of the process according to the invention can be monitored. With such monitoring, the skilled person is able to identify those substances which exhibit the inhibition of said proteases according to the invention.

In particular, the process according to the invention is characterised in that cells are cultivated which express a fusion protein which contains the specific cleavage site of γ-secretase (see also Example 1). The fusion protein with the γ-secretase substrate according to the invention may be membrane associated in general terms but is preferably membrane-based.

Moreover, the process according to the invention is characterised in that cells are cultivated which express a fusion protein containing the specific cleavage site of presenilinase.

In a particular embodiment, the process according to the invention is also characterised in that the fusion protein contains amyloid β. This is the substrate of γ-secretase.

For a particular embodiment of the process according to the invention, the fusion protein contains a fragment of amyloid β. This represents the substrate for the γ-secretase and according to the invention must contain at least one specific γ-secretase cutting site.

For one particular embodiment of the process according to the invention, the fusion protein contains the amyloid precursor protein or a fragment thereof. For example, the fusion protein may consist of a reporter protein and the C99 fragment. The DNA which codes for the γ-secretase substrate or for the C99 fragment can be obtained from suitable gene banks using standard methods. This constitutes the substrate for the γ-secretase and according to the invention must contain at least one specific γ-secretase cutting site.

For another particular embodiment of the process according to the invention, the fusion protein comprises presenilin 1 or a fragment thereof. The fragments may be, for example, N-terminal fragments (NTF) of about 21-28 kDa or C-terminal fragments (CTF) of 16-24 kDa (Haass, C. et al., J. Neurol. Transm. Suppl., 1998, 53:159-167; Okochi, M. et al., 1997, FEBS Lett., 1997, 418:162-166; Borchelt, D. R. et al., Neuron, 1996, 17:181-190), which must nevertheless go beyond the presenilase cutting site. Presenilin 1 or the fragments thereof constitute the substrates of the presenilinase and according to the invention must contain at least one specific presenilinase cutting site.

In another particular embodiment of the process according to the invention, the fusion protein comprises presenilin 2 or a fragment thereof. The fragments may for example be N-terminal fragments (NTF) of about 28-30 kDa (NTF) kDa or C-terminal fragments (CTF) of 20-25 kDa (Haass, C. et al., J. Neurol. Transm. Suppl.,1998, 53:159-167; Kim, T. W. et al., J. Biol. Chem., 1997, 272:11006-11010; Podlisny, M. B. et al., Neurobiol. Dis., 1997, 3:325-337), which must go beyond the presenilinase cutting site. Presenilin 2 or the fragments thereof constitute the substrates for the presenilinase and according to the invention must contain at least one specific presenilinase cutting site.

Preferably, the process according to the invention is a high throughput screening (HTS) test. This HTS has advantages over the prior art since it allows a number of substances to be tested for their effectiveness by a simple method. A high throughput test or HTS is a method of investigating a large to very large number of substances simultaneously.

Preferably, an HTS may be carried out in microtitre plates, it may be partially or totally automated and may be connected to electronic equipment such as computers for data storage, analysis and evaluation using bioinformatics. Preferably, this automation makes use of both robots, as used in the automobile industry, which are capable of handling large numbers of microtitre plates simultaneously or sequentially, and also automatic pipetting machines. These automatic or semi-automatic HTS systems are capable of performing several thousand tests a day and are commercially available. Test substances can be acquired commercially as libraries of substances. The test substances are preferably dissolved in an organic solvent and are most preferably dissolved in DMSO. Preferably, the test substances which exhibit the desired protease inhibitor function in a cell-free system are also tested in a cell-based system. The term high throughput test or HTS also includes ultra-high throughput screening: UHTS. Preferably, these ultra-high throughput tests or UHTS are carried out in 384- or 1536-well microtitre plates with sub-microliter or sub-nanoliter pipettors, improved plate reading equipment and processes which prevent evaporation. HTS methods are described, for example, in U.S. Pat. Nos. 5,876,946 A or 5,902,732 A. The average person skilled in the art will be aware of other literary sources on HTS tests and can adapt the processes of the present invention to an HTS or UHTS format without any inventive step and without having to be inventive themselves. Within the scope of this invention, a reporter protein should be taken to mean a protein having the capability of generating an easily detected signal the quantity of which correlates with the quantity of the cleavage product in question. The signal is generated either by the measurement of the enzymatic activity of the reporter protein with substances which are easily detected or by measuring the intensity of fluorescence of the reporter protein. Examples of reporter proteins are the green fluorescent protein (GFP) (see for example WO95/07463) or derivatives thereof which fluoresce at other wavelengths or enzymes such as luciferase, secretory alkaline phosphatase or β-galactosidase.

Therefore, according to another preferred embodiment of the process according to the invention, the quantity of reporter cleaved is measured with luciferase, green fluorescent protein or a derivative thereof, secretory alkaline phosphatase or β-galactosidase. In yet another preferred embodiment of the process according to the invention the quantity of reporter cleaved is detected indirectly.

A large number of transcription factors are known, which require two subunits for their activity. In other cases, a single transcription factor may require two separate functional domains for its activity (e.g. one transcriptional activator domain and one DNA binding domain) so that each domain on its own is inactive but when they are brought together the transcriptional activity can take place.

One of these factors is Gal4 which, as described, can be divided into two domains (Laughan, A. and Gesteland, R., Molec. Cell Biol., 1984, 4: 260-267; Fields, S. and Song, O., Nature, 1989, 340: 245-246). Other transcription factors are members of the Jun, Fos and ATF/CREB families, Oct1, Sp1, HNF-3, the steroid receptor superfamily and others. Similarly, in yet another preferred embodiment of process according to the invention, the quantity of reporter cleaved can be detected indirectly when a cleaved reporter domain migrates into the nucleus, binds to another reporter domain of a reporter construct therein and the expression of the reporter is activated.

In yet another preferred embodiment of the process according to the invention, the quantity of reporter Gal4 cleaved is detected indirectly when Gal4 in the nucleus binds to the Gal4 DNA binding domain of another reporter construct and the expression of luciferase is activated (see Examples 2 and 4).

In yet another preferred embodiment of the process according to the invention the quantity of reporter cleaved is detected directly. For example, this might be direct detection of the cleaving by the protease by means of a reporter which is only activated when cleaving occurs. Examples of reporters may be those described above: GFP, or enzymes such as luciferase, secretory alkaline phosphatase or β-galactosidase.

The invention may be performed using any cells or cell lines known in the art, particularly eukaryotic cells or cell lines. Preferred cells or cell lines are those used in neurological or neurobiological research, e.g., mammalian cells or cell lines such as H4, U373, NT2, HEK 293, PC12, COS, CHO, fibroblasts, myeloma cells, neuroblastoma cells, hybridoma cells, oocytes, embryonic stem cells. It is also possible to use insect cell lines (e.g. using Baculovirus vectors such as pPbac or pMbac (Stratagene, La Jolla, Calif.)), yeast (e.g. using yeast expression vectors such pYESHIS (Invitrogen, CA)) and fungi.

Particularly preferred are cells or cell lines of neuronal or glial origin. In a most particularly preferred embodiment of the invention, the cells used are H4 cells, human neuroglioma cells from the brain which were deposited under ATCC Number HTB-148 at the American Type Culture Collection (ATCC) in Manassas, Va., USA. Therefore, in a special embodiment of the process according to the invention, the cells are of neuronal or glial origin. In one particular embodiment of the process according to the invention the cells are H4 cells.

According to the invention, a high throughput screening test for finding substances capable of specifically inhibiting γ-secretase is characterised in that:

a) H4 cells are cultivated which endogenously or exogenously have said γ-secretase activity and which express the membrane associated recombinant fusion protein Aβ-KKK/Gal4;

b) these cells are incubated with a test substance;

c) the quantity of transactivator Gal4 cleaved and binding to the Gal4 DNA binding domain of the luciferase construct is measured by means of the level of luciferase expression; and

d) the value obtained is compared with the value obtained in the absence of the test substance.

This high throughput screening test according to the invention is described in more detail in the Examples.

The processes or high throughput screening tests according to the invention may be used within the scope of the invention to find substances capable of specifically inhibiting γ-secretase.

The processes or high throughput screening tests according to the invention may be used within the scope of the invention to find substances capable of specifically inhibiting presenilinase.

The invention also includes substances which can be found using a process or high throughput screening test according to the invention, characterised in that they specifically inhibit the proteolytic cleaving of a γ-secretase substrate. For example, these substances may be proteins or chemical compounds. A substance according to the invention is substance A as shown in the following formula (see also Example 8):

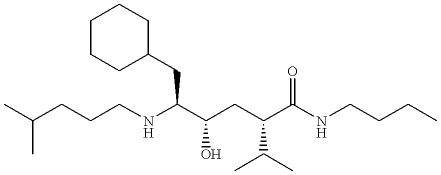

The invention further relates to substances which can be found using a process or high throughput screening test according to the invention, characterised in that they specifically inhibit the proteolytic cleaving of a presenilinase substrate. These substances may be proteins or chemical compounds, for example. Said substances according to the invention may be used within the scope of this invention for preparing a medicament for treating neurodegenerative diseases, particularly Alzheimer's disease. Other neurodegenerative diseases include, for example, Alzheimer's, Parkinson's and Huntington's chorea. The invention also relates to a pharmaceutical formulation which contains a substance according to the invention as well as conventional pharmaceutical carriers.

A pharmaceutically acceptable carrier may contain physiologically acceptable compounds which increase the stability or absorption of the substance according to the invention, for example. Such physiologically acceptable compounds contain, for example, carbohydrates such as glucose, saccharose or dextrans, antioxidants such as ascorbate or glutathione, chelating agents, proteins of low molecular weight or other stabilisers (cf. for example Remington's Pharmaceutical Sciences (1990)). Anyone skilled in the art knows that the choice of a pharmaceutically acceptable carrier including a physiologically acceptable compound depends on the route of administration, for example.

The Examples which follow are intended to illustrate the invention without restricting its scope in any way.

### EXAMPLE 1

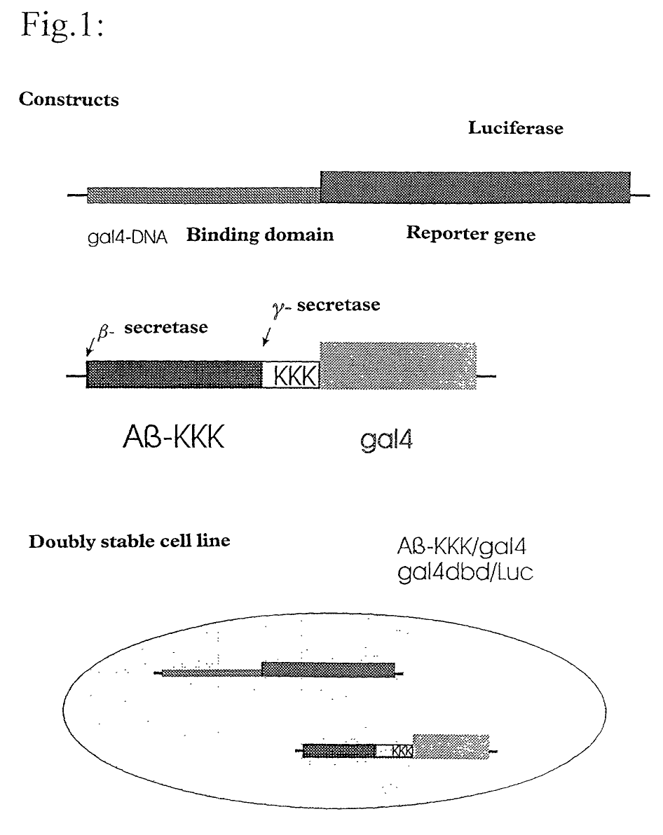

### Preparation of Reporter Constructs and Cell Lines (see also FIG. 1)

Carrying out the Test System:

1.1 Preparing a Suitable Cell Line γ-secretase HTS:

H4 neuroglioma cells (Accession Number HTB 148 at the American Type Culture Collection, Manassas, Va., USA) were stably transfected under standard conditions with the reporter construct pFRLuc (Stratagene) which carries the gene for luciferase which is under the control of a promoter containing the Gal4 DNA binding domain. By transient transfection experiments with pcDNA3-Gal4 which codes for soluble Gal4 an individual clone was selected which showed the highest luciferase activity. In order to prepare the Aβ-KKK/Gal4 construct, a sequence which contained the N-terminal signal sequence of APP and the first 55 amino acids of Aβ (Shoji, M. et al., Science, 1992; 158:126-129) was attached to the Gal4 coding sequence (Laughan, A. and Gesteland, R., Molec. Cell Biol., 1984, 4:260-267) by genetic engineering methods and cloned into the expression vector pcDNA3neo (Invitrogen). This construct was designated Aβ-KKK/Gal4. In order to insert an ER-retention signal in the substrate of the γ-secretase, the last nucleotides of the Gal4 were modified by genetic engineering methods so that they coded for amino acids KKLI. This construct was named Aβ-KKK-ER/Gal4. The cell clone obtained as described above was used for the second stable transfection with pcDNA3/Aβ-KKK/Gal4 or pcDNA3/Aβ-KKK-ER/Gal4 and the selection was carried out with neomycin under standard conditions (Sambrook and Maniatis, Molecular Cloning: A Laboratory Manual, Cold Spring Harbor Laboratory, Cold Spring Harbor, N.Y., 1989). Individual neomycin-resistant cell clones were tested for expression of luciferase. The clones with the highest expression were used for the substance analyses. All the transfections were carried out using the Fugene transfection system of Boehringer Mannheim in accordance with the manufacturer's instructions.

1.2 Preparation of a Suitable Construct for Presenilinase HTS:

In order to identify inhibitors of presenilinase, a construct was designed consisting of a substrate portion (presenilin part) and a reporter part (Gal4). The presenilin part consists of the nucleotides which code for amino acids 3-101 and amino acids 263 to 325 of PS-1 (Sherrington, R. et al., Nature, 1995, 375:754-760). The first region contains the first transmembrane region of PS-1. By gene manipulation methods, the human APP signal sequence (Shoji, M. et al., Science, 1992, 126:126-129) was attached to the N-terminus of the first presenilin sequence. The reporter fragment adjoins the second presenilin region (Gal4, amino acid 1-881; Laughan, A. and Gesteland, R., Molec. Cell Biol., 1984, 4: 260-267). This construct was cloned into the expression vector pcDNA3neo (Invitrogen).

### EXAMPLE 2

### Carrying out the Test System

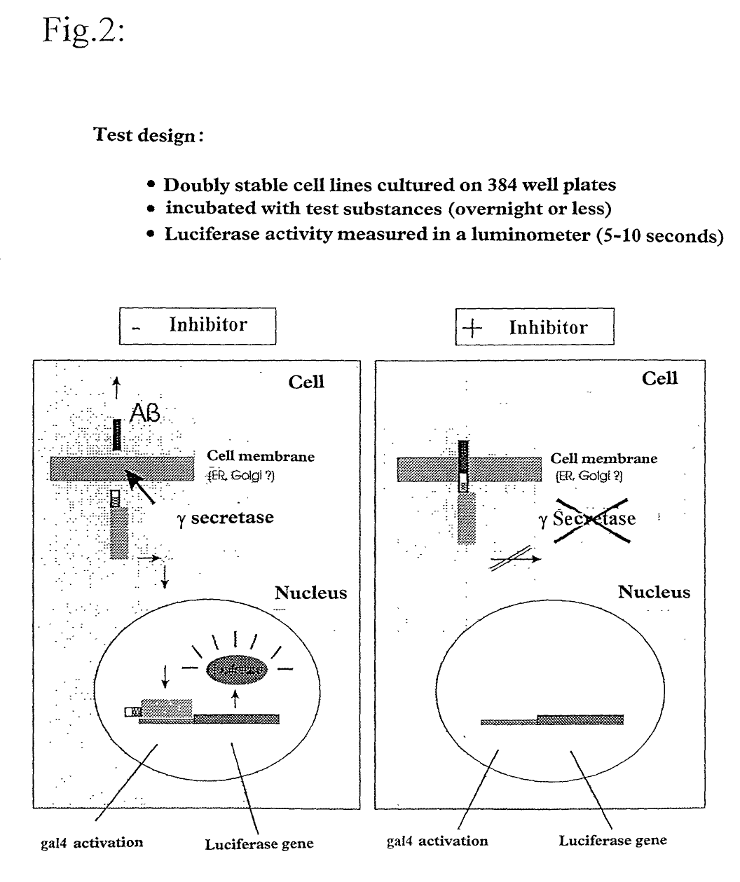

HTS Assay Principle (see also FIG. 2).

The doubly stable HTS cell line is seeded onto a 96/384 microtitre plate. When the cells achieved confluence they are incubated with the test substance in question for a specified length of time. After incubation the cell medium is removed and the luciferase enzyme activity is determined precisely in accordance with the instructions given by the manufacturer of the test kit used (SteadyGlo, Promega or a different comparable manufacturer).

By the presence of an endogenous or exogenous γ-secretase activity in the H4 cells, the substrate (Aβ-KKK/Gal4) is cleaved proteolytically, whereupon the transactivator Gal4 can detach itself from the membrane and enter the cell nucleus. In the cell nucleus, Gal4 binds to the Gal4 DNA binding domain of the reporter construct and thus activates the expression of the luciferase. When a specific γ-secretase inhibitor is present the substrate cannot be cleaved and Gal4 remains bound to the substrate on the cell membrane, leading to a reduction and even total inhibition of the luciferase activity.

Cells are seeded onto 96/385 well plates in DMEM whole medium (10% FCS, 1% glutamine, 1% penicillin/streptomycin) in a dilution of 1:5. The cells are incubated for 24 to 48 hours (depending on the cell clone and dilution used) at 37° C. in 5% CO₂ and allowed to grow until 80-90% confluence is achieved (which may take less or more time). Then the test substance is added and incubated overnight (8-16 hours) at 37° C. and 5% CO₂. The 96/384 well plates are equilibrated at room temperature (RT). The SteadyGlo Luciferase Assay System (Promega Catalogue No.E2520) is used. The luciferase assay reagent is thawed and equilibrated at ambient temperature or used fresh (luciferase assay substrate dissolved in luciferase assay buffer). The medium is suction filtered from the cells. One hundred microliters (based on 96 well plates) of fresh whole medium are added to each well. One hundred microliters (based on 96 well plates) of luciferase assay reagent are added and incubated for 5 minutes at RT. Then, the luminescence is measured. For 384 well plates the amounts pipetted are reduced accordingly, in a manner known in the art.

### EXAMPLE 3

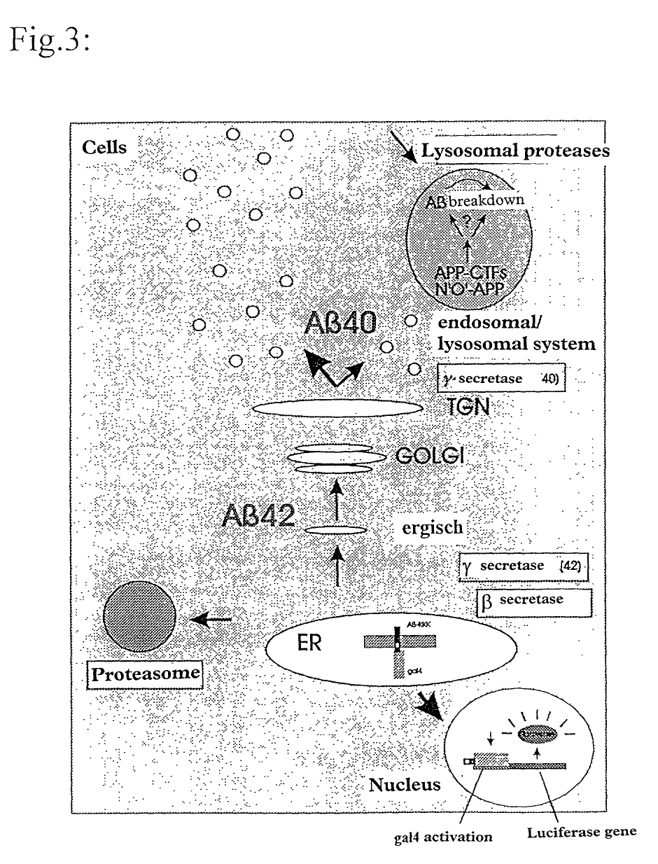

### ER Retention of the γ-Secretase Substrate—Intracellular APP Processing (see FIG. 3).

APP is a transmembrane protein which is processed along the secretory metabolic route. The full length protein is presumably already cleaved in very early cell compartments such as ER (endoplasmic reticulum) and Golgi apparatus by the so-called β- and γ-secretases, forming predominantly Aβ42 (Wild-Bode C. et al., J. Biol. Chem., 1997, 272:16085-16088). In the trans Golgi network (TGN), in the vessels tied off by the TGN and migrating to the cell membrane, or on the cell membrane directly, the Aβ40 is presumably formed by the activity of the same or similar proteases (Borchelt, D. R. et al., Neuron., 1996, 17:181-190). APP which has not been processed by the secretory metabolic route can enter the endosomal/lysosomal system by endocytosis and there be degraded to Aβ or degraded fully (Koo, E. H. and Squazzo, S. L., J. Biol. Chem., 1994, 269:17386-17389).

In order to increase the concentration of substrate in those cellular compartments in which the formation of Aβ42 takes place and in order to rule out the identification of non-specific inhibitors such as transporting inhibitors, an ER retention signal sequence was appended to the γ-secretase substrate, the Aβ-KKK/Gal4 fusion protein.

### EXAMPLE 4

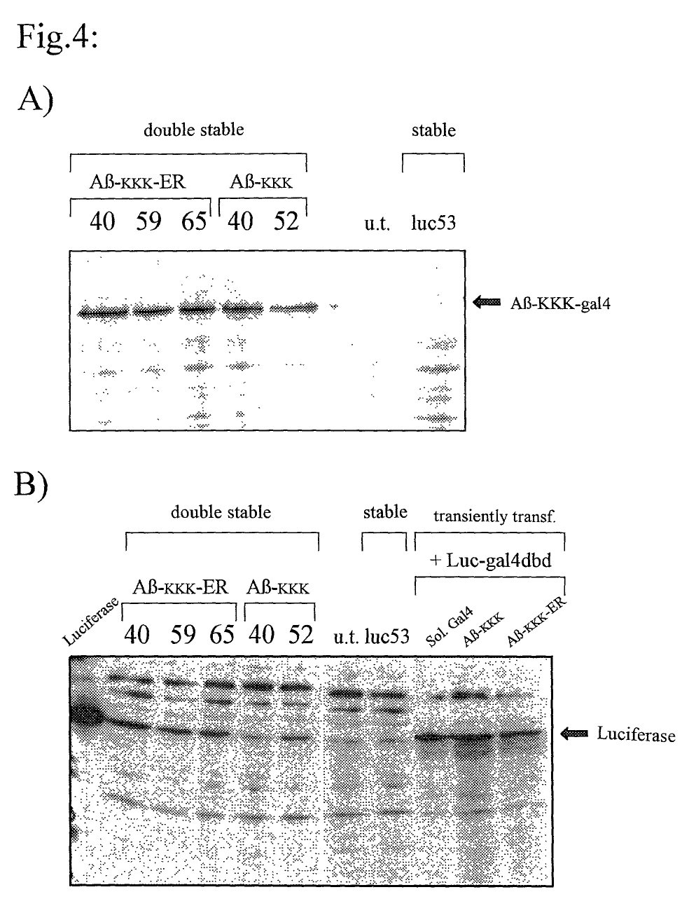

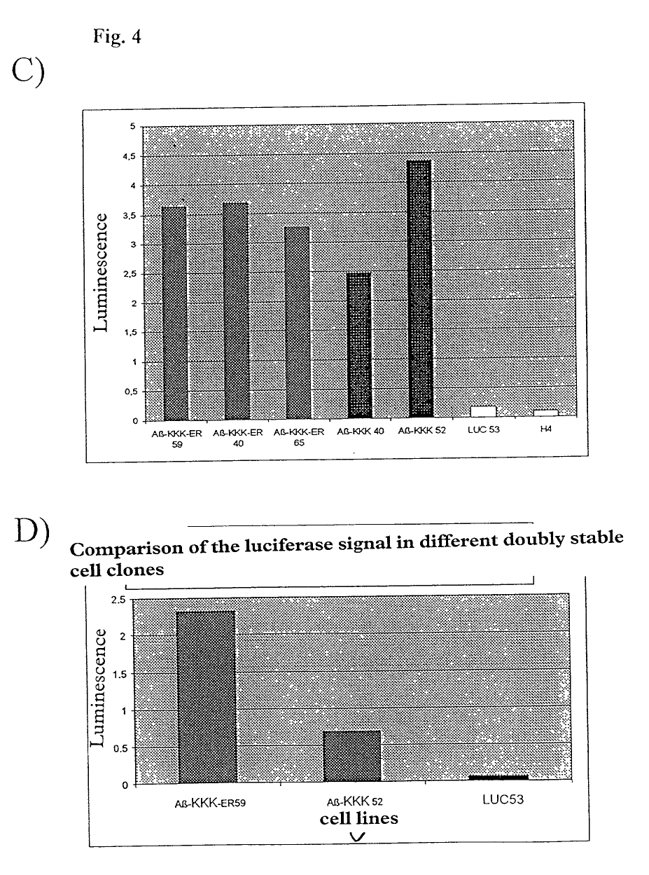

### Characterisation of the Transfected Cell Lines for HTS (see FIGS. 4A-4D).

A) Expression of the Aβ-KKK/Gal4 Substrate.

A fairly large number of different doubly stable H4 cell clones were prepared and investigated for the expression of the Aβ-KKK/Gal4 substrate protein. Cell extracts were prepared by standard methods, the proteins were separated in a 10% SDS polyacrylamide gel and transferred onto a PVDF membrane. The fusion protein was detected by chemiluminescence with a primary antibody against Gal4 (Clontech; 1:5000) and with a secondary Horseradish Peroxidase-coupled Antibody (Amersham; 1:6000).

By way of example, the expression of the fusion protein is shown here in cell clones Aβ-KKK-ER/40, 59 and 65 (substrate Aβ-KKK/Gal4 with ER retention signal) and in the cell clones Aβ-KKK/40 and 52 (substrate Aβ-KKK/Gal4 without ER retention signal). By contrast, no expression could be detected in the untransfected H4 cells (u.t.) and in the H4-Luc53 cells (luc53) which were transfected only with a reporter construct Gal4dbd-Luc (Gal4-DNABinding domain/luciferase; in English luciferase).

B) Determining Luciferase Expression by Western Blot.

The cell clones Aβ-KKK-ER/40, 59 and 65 (substrate Aβ-KKK/Gal4 with ER retention signal) and the cell clones Aβ-KKK/40 and 52 (substrate Aβ-KKK/Gal4 without ER retention signal) were investigated for the expression of luciferase. Cell extracts were prepared by standard methods, the proteins were separated in a 10% SDS polyacrylamide gel and transferred onto a PVDF membrane. The luciferase was detected by chemiluminescence with a primary antibody (Europa) in a 1:100 dilution and with a secondary horseradish peroxidase-coupled antibody (Amersham; 1:6000). The luciferase expression could be detected in all the doubly stable cell clones but not in the untransfected H4 (u.t.) and the H4-Luc53 cells (luc53). As a positive control, H4 cells were co-transfected with the various substrates (soluble gal4 [sol. gal4], Aβ-KKK/Gal4 and Aβ-KKK-ER/Gal4) and the reporter construct Gal4dbdLuc (Gal4-DNA-Binding domain/luciferase; in English luciferase). In these transient transfections, too, the luciferase could be detected in the H4 cells.

C) Determining Luciferase Expression by Enzymatic Detection.

The expression level of luciferase in the cell clones Aβ-KKK-ER/40, 59 and 65 (substrate Aβ-KKK/Gal4 with ER retention signal) and in the cell clones Aβ-KKK/40 and 52 (substrate Aβ-KKK/Gal4 without ER retention signal) was also investigated by enzymatic activity measurement (SteadyGlo, Promega). The luciferase assay was carried out precisely according to the instructions of the manufacturer (Promega). Luciferase activity could be detected in all the doubly stable cell clones but not in the untransfected H4 (H4) and in the H4-Luc53 cells (luc53). In this test the signal was not related to the amount of protein.

D) Determining the Luciferase Expression by Enzymatic Detection.

The luciferase activity was determined in the two cell clones Aβ-KKK-ER/59 (substrate Aβ-KKK/Gal4 with ER retention signal) and Aβ-KKK152 (substrate Aβ-KKK/Gal4 without ER retention signal). The luciferase assay (SteadyGlo, Promega) was carried out exactly as instructed by the manufacturer (Promega).

Luciferase activity could be detected in all the doubly stable cell clones but not in the untransfected H4 (H4) or in the H4-Luc53 (luc53) cells. In this experiment the signal was related to the amount of protein. The differences in signal intensity between the two cell clones correlated with the expression level of the substrate (see FIG. 4A).

### EXAMPLE 5

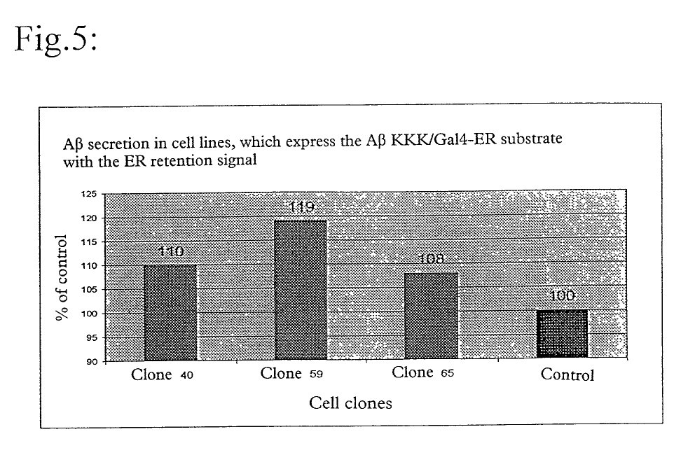

### Determining the Levels of Extracellular Aβ40 and Aβ42 in the Transfected HTS Test Cell Lines (see FIG. 5)

The cell clones Aβ-KKK-ER/40, 59 and 65 (substrate Aβ-KKK/Gal4 with ER retention signal) and the cell clones Aβ-KKK/40 and 52 (substrate Aβ-KKK/Gal4 without ER retention signal) were tested for their level of extracellular Aβ40 and Aβ42. Fresh medium was added to the confluent cells and these were incubated for a further 16 hours. After incubation the medium was removed and the concentration of Aβ40 and Aβ42 was determined in a quantitative ELISA (Steiner, H. et al., J. Biol. Chem., 1998, 273:32322-32331). Untransfected H4 cells were used as the control, and the concentration of secreted Aβ40 and Aβ42 was put at 100%.

The levels of extracellular Aβ42 in the cell clones Aβ-KKK-ER/40, 59 and 65 were slightly raised, but no difference could be detected for the quantity of secreted Aβ40 in these clones. A higher concentration could not be found for cell clone Aβ-KKK/40 and 52 either for Aβ40 or for Aβ42.

### EXAMPLE 6

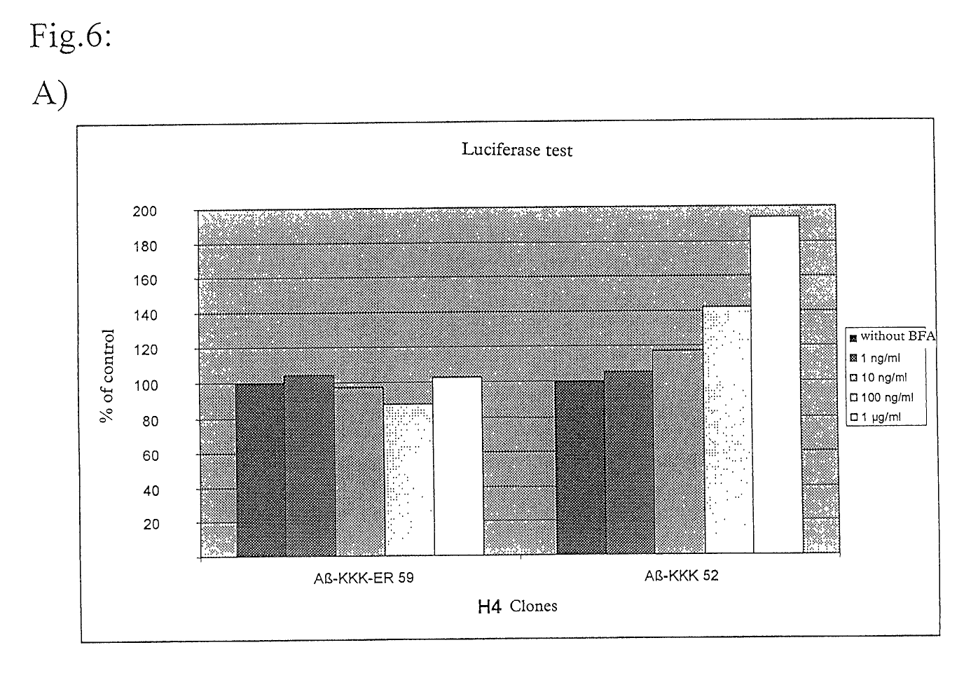

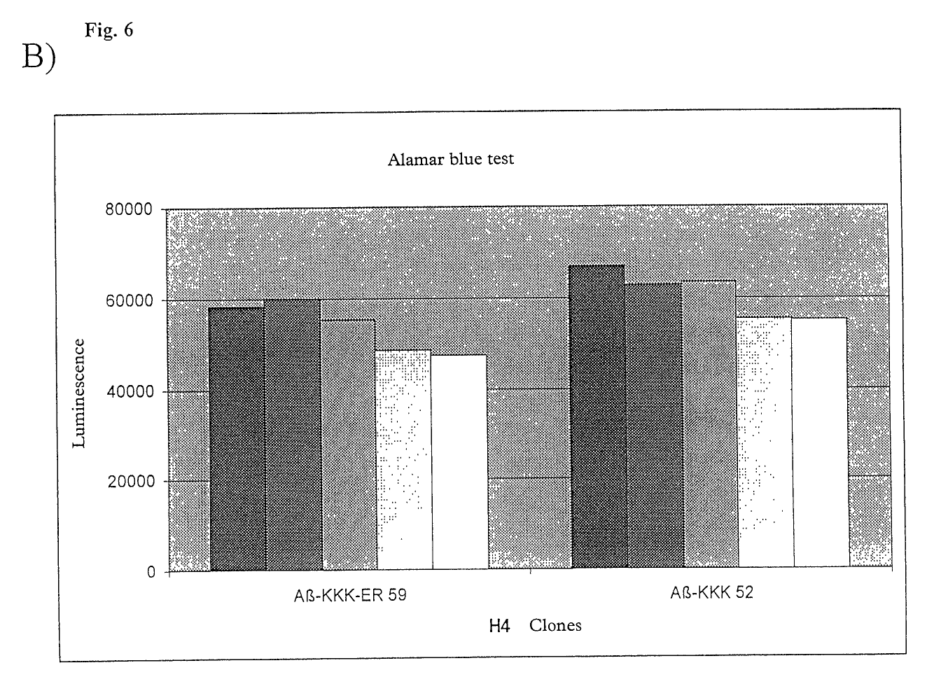

### BFA Effect in the HTS Test Cell Lines Aβ-KKK-ER/59 and Aβ-KKK/52 (see FIGS. 6A-6B).

Cells of the clones Aβ-KKK-ER/59 (substrate Aβ-KKK/Gal4 with ER retention signal) and Aβ-KKK/52 (substrate Aβ-KKK/Gal4 without ER retention signal) were treated for 6 hours with different concentrations of Brefeldin A (BFA). Untreated cells were used as the control.

A) Luciferase Test.

The luciferase was detected by enzymatic activity measurement. The luciferase assay was carried out exactly as prescribed by the manufacturer (SteadyGlo, Promega). A clear BFA effect could be seen in cell clone Aβ-KKK/52 but not in cell clone Aβ-KKK-ER/59. An increase in the BFA concentration lead to an increased Aβ formation in the ER (Wild-Bode, C. et al., J. Biol. Chem., 1997, 272:16085-16088) and hence to an increase in the luciferase signal.

B) Vitality Test.

A possible toxic effect of the BFA (in different concentrations) on the cells was investigated using an Alamar Blue test (see below) which was carried out in parallel to the luciferase test. No loss of vitality could be found over the entire range of concentrations.

Alamar Blue Measurement (Cytotoxicity Measurement)

Preparation:

Alamar Blue (Messrs. Biosource International, VE=100 ml, Cat.No. DAL 1100 (AL-01 -100)) was sterile-filtered. The seeding of the cells and substance incubation were carried out as in Example 1.

The Alamar blue incubation was carried out parallel to the luciferase test.

Method:

Alamar blue was diluted to 5% with whole medium (DMEM m 4.5 g/l glucose+10% FCS+1% glutamine+1% Pen/Strep). The medium was suction filtered. The cells were washed once with medium or PBS. One hundred microliters of (5%) Alamar blue were added (96 well plate) and a plate was incubated for 1 hour in the incubator at 37° C. and in 5% CO2. Fluorimetric measurement was carried out at an input wavelength of 544 nm, an emission wavelength of 590 nm and a measuring time of 0.3 s/well. The results are given as a percentage of the control.

### EXAMPLE 7

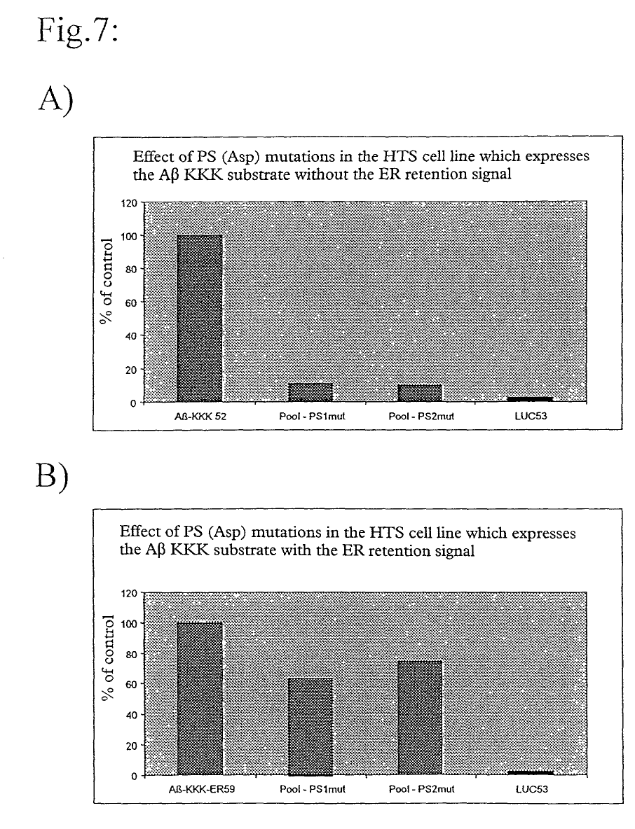

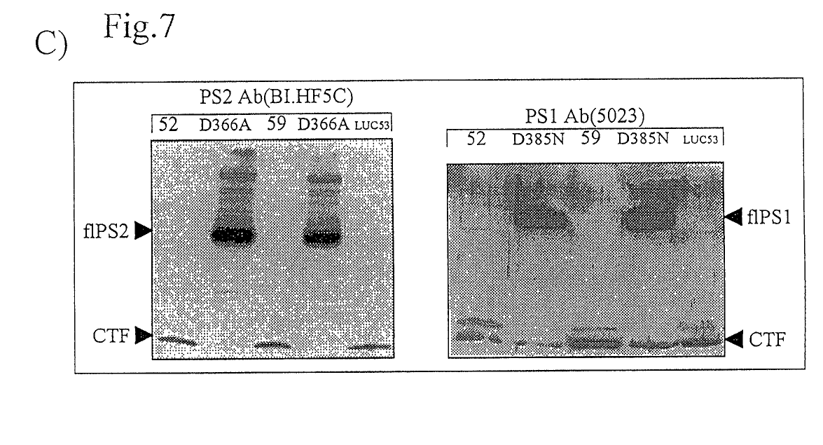

### Effect of Presenilin (PS1 and PS2) Aspartate Mutations in the HTS Test Cell Lines Aβ-KKK-ER/59 and Aβ-KKK/52 (see FIGS. 7A-7C).

The cell clones Aβ-KKK-ER/59 (substrate Aβ-KKK/Gal4 with ER retention signal) and Aβ-KKK/52 (substrate Aβ-KKK/Gal4 without ER retention signal) were transfected with cDNAs which coded for PS1 or PS2 and carried an aspartate(Asp) mutation (PS1-D385N or PS2-D366A). Forty-eight hours after transfection the cells were selected with zeozin in order to obtain pool cells.

A) Effect of PS (Asp) Mutants in the Pool Cells Aβ-KKK/52/D385N and Aβ-KKK/52/D366A.

The expression of the luciferase in the Aβ-KKK/52 (substrate Aβ-KKK/Gal4 without ER retention signal) pool cells pool-PS1mut (D385N) and pool-PS2mut (D366A) was investigated by enzymatic activity measurement. The luciferase assay was carried out precisely in accordance with the instructions of the manufacturer (SteadyGlo, Promega). A significant reduction in the luciferase signal to approximately 10% of the control value could be detected in both pool cells. The H4-Luc53 cells were used as a control.

B) Effect of the PS (Asp) Mutants in the Pool Cells Aβ-KKK-ER/59/D385N and Aβ-KKK-ER/52/D366A.

The expression of the luciferase in the Aβ-KKK-ER/59 (substrate Aβ-KKK/Gal4 without ER retention signal) pool cells pool-PS1mut (D385N) and pool-PS2mut (D366A) was investigated by enzymatic activity measurement. The luciferase assay was carried out precisely in accordance with the instructions of the manufacturer (SteadyGlo, Promega). A reduction in the luciferase signal to approximately 60% (PS1mut), or approximately 70% (PS2mut) of the control could be detected in both pool cells. The H4-Luc53 cells were used as a control.

C) Detecting the Overexpression of PS (Asp) Mutants in the Cell Lines.

The pool cells Aβ-KKK/52/D385N and D366A and the pool cells Aβ-KKK-ER/59/D385N and D366A were investigated for overexpression of the presenilin aspartate mutant by Western blot methods. Cell extracts were prepared by standard methods; the proteins were separated in a 12% SDS-polyacrylamide gel and transferred onto a PVDF membrane. PS1 was detected with a polyclonal antibody (5023; 1:1000; Walter, J. et al., Proc. Natl. Acad. Sci. USA, 1997, 94:5349-5354) and PS2 with a monoclonal antibody (BI.HF5C; 1:3000; Steiner, H. et al., Biochem., 1999b, 38:14500-14505). As described (Steiner, H. et al., J. Biol. Chem., 1999a, 274:7615-7618; Wolfe, M. S. et al., Nature, 1999, 398:513-517), both aspartate mutations prevent the proteolysis of PS proteins so that only full-length PS can be detected. The overexpression of the exogenous PS moreover leads in every case to a significant reduction in the endogenous PS, which is apparent from the reduction in PS-C-terminal fragments (CTFs).

### EXAMPLE 8

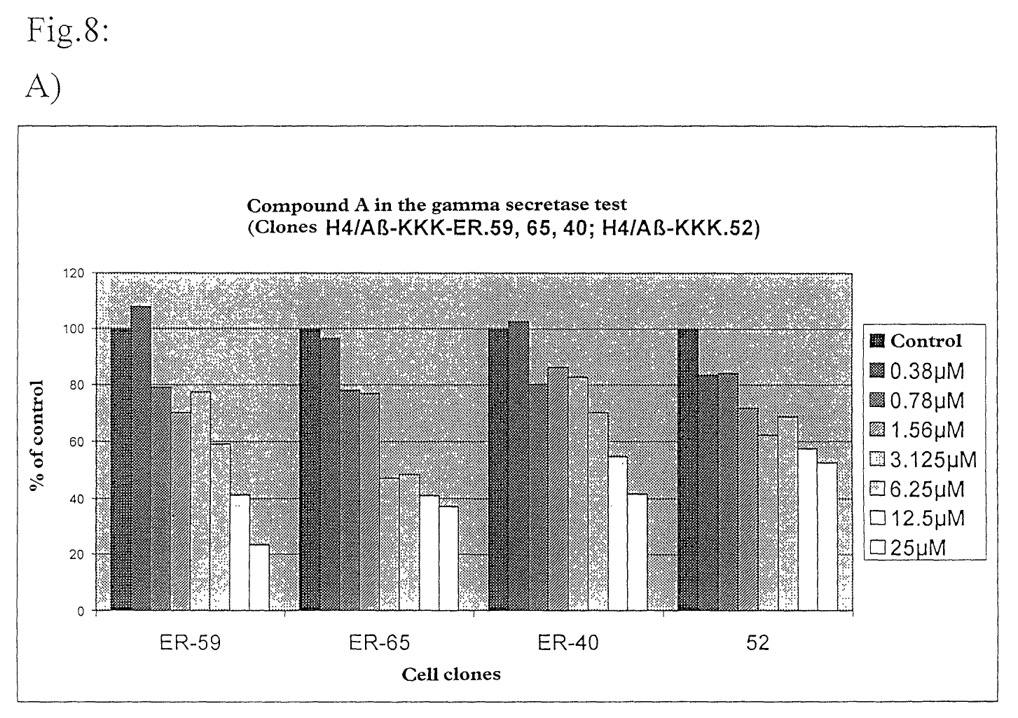

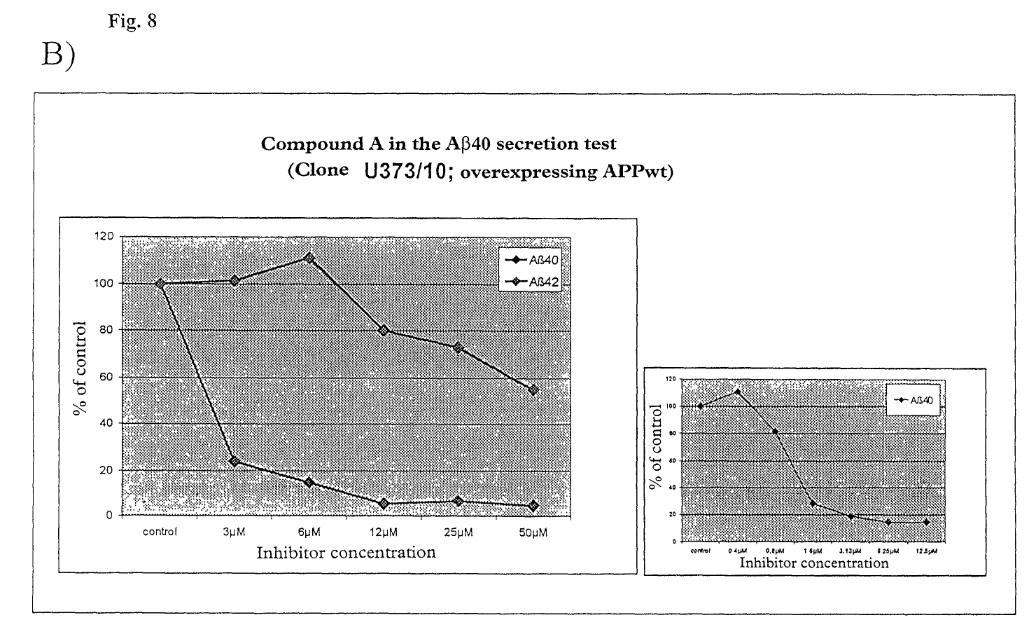

### Substance A. (see FIGS. 8A-8B)

A) Concentration-dependent Inhibition of Aβ Formation by Substance A.

Substance A:

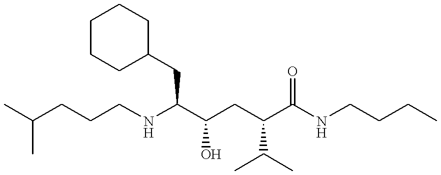

The cell clones Aβ-KKK-ER/40, 59 and 65 (substrate Aβ-KKK/Gal4 with ER retention signal) and the cell clone Aβ-KKK/52 (substance Aβ-KKK/Gal4 without ER retention signal) were used to test the inhibitory effect of substance A. Confluent cells were incubated overnight with different concentrations of substance A. The luciferase assay was carried out exactly in accordance with the manufacturer's instructions (SteadyGlo, Promega).

In all cell clones, substance A inhibits Aβ formation in a manner dependent on concentration, as indicated by the decrease in the luciferase signal.

B) Concentration Dependent Inhibition of Aβ40 and Aβ42 Secretion by Substance A.

Substance A was also active in a cellular test system in which the extracellular content of Aβ40 and Aβ42 secreted by the U373 cell line (U373: ATCC No. HTB 14) was determined. This cell line, U373/APP751, is an astrocytoma cell line which overexpresses human APP751 and secretes large amounts of Aβ40 (about 1000 pg/ml/4 hours with 5×10⁷ cells in 15 ml of medium) and Aβ42 (about 1000 pg/ml/4 hours with 5×10⁷ cells in 15 ml of medium). The measurement is carried out by ELISA (Steiner, H. et al., J. Biol. Chem, 1998, 273:32322-32331). The Aβ40 secretion was greatly reduced in concentration-dependent manner by substance A, whilst the Aβ42 secretion was increased at sub-inhibitory doses and then also inhibited at higher doses.

### EXAMPLE 9

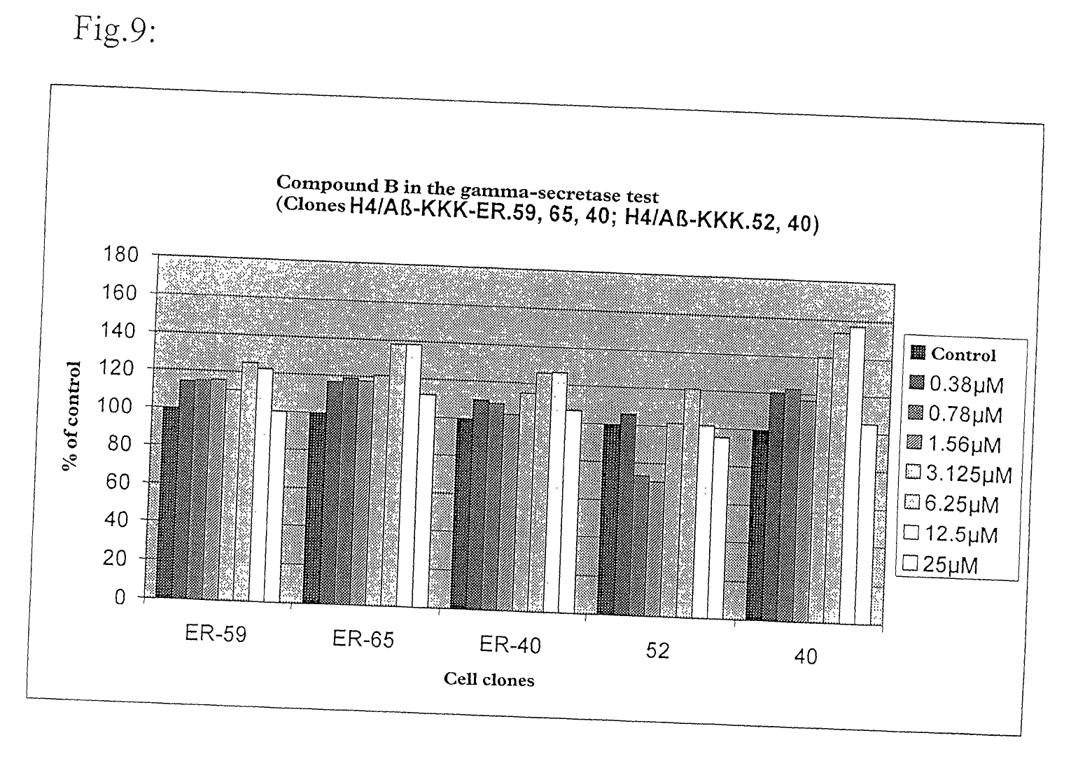

### Substance B (see FIG. 9)

Effect of Substance B on Aβ Formation.

Substance B:

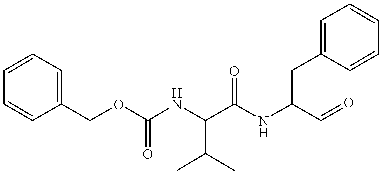

Cell clones Aβ-KKK-ER/40, 59 and 65 (substrate Aβ-KKK/Gal4 with ER retention signal) and cell clones Aβ-KKK/40 and 52 (substrate Aβ-KKK/Gal4 without ER retention signal) were used to test the inhibitory effect of substance B. Confluent cells were incubated overnight with different concentrations of substance B. The luciferase assay was carried out precisely in accordance with the manufacturer's instructions (SteadyGlo, Promega).

Substance B does not inhibit Aβ formation in concentration-dependent manner in any of the cell clones, as demonstrated by the constant signal intensity of the luciferase.

### EXAMPLE 10

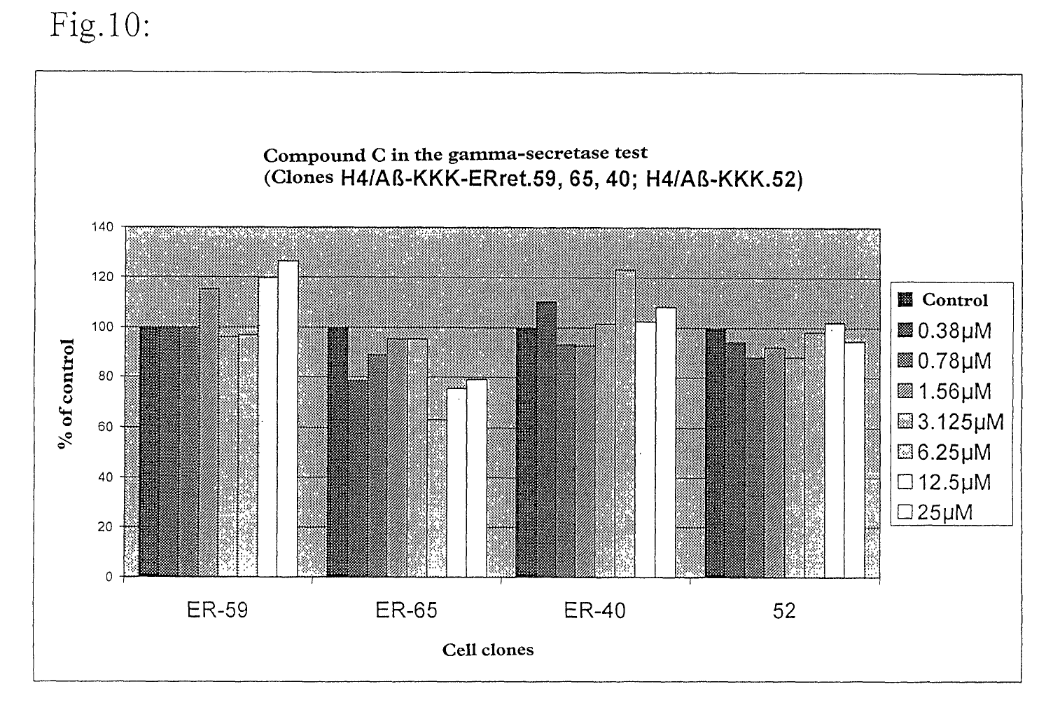

### Substance C (see FIG. 10)

Effect of Substance C on Aβ Formation.

Substance C:

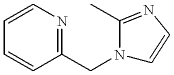

Cell clones Aβ-KKK-ER/40, 59 and 65 (substrate Aβ-KKK with ER retention signal) and cell clone Aβ-KKK/52 (substrate Aβ-KKK without ER retention signal) were used to test the inhibitory effect of substance C. Confluent cells were incubated overnight with different concentrations of substance A. The luciferase assay was carried out precisely in accordance with the manufacturer's instructions (SteadyGlo, Promega). Substance C does not inhibit Aβ formation in concentration-dependent manner in any of the cell clones, as demonstrated by the constant signal intensity of the luciferase.

The present invention is not to be limited in scope by the exemplified embodiments which are intended as illustrations of single aspects of the invention. Indeed various modifications of the invention in addition to those shown and described herein will become apparent to those skilled in the art from the foregoing description and accompanying drawings. Such modifications are intended to fall within the scope of the appended claims.

All publications and patent applications cited herein are incorporated by reference in their entireties.

What is claimed is:

1. A method for identifying substances which are capable of specifically inhibiting proteases which cleave membrane-bound substrates comprising:
a) cultivating cells which exhibit said protease activity and express a membrane-associated recombinant fusion protein, said fusion protein comprising: (i) the substrate of said protease containing the specific cleavage site for said protease and (ii) a reporter;
b) incubating said cells with a test substance;
c) measuring the quantity of reporter cleaved; and
d) comparing the value obtained in step (c) with the value obtained in the absence of the test substance.

2. The method according to claim 1, wherein the fusion protein comprises the specific cleavage site of γ-secretase.

3. The method according to claim 1, wherein the fusion protein comprises the specific cleavage site of presenilinase.

4. The method according to claim 1, wherein the fusion protein comprises amyloid β or a fragment thereof.

5. The method according to claim 2, wherein the fusion protein comprises amyloid β or a fragment thereof.

6. The method according to claim 1, wherein the fusion protein comprises the amyloid precursor protein or a fragment thereof.

7. The method according to claim 2, wherein the fusion protein comprises the amyloid precursor protein or a fragment thereof.

8. The method according to claim 1, wherein the fusion protein comprises presenilin 1 or a fragment thereof.

9. The method according to claim 3, wherein the fusion protein comprises presenilin 1 or a fragment thereof.

10. The method according to claim 1, wherein the fusion protein comprises presenilin 2 or a fragment thereof.

11. The method according to claim 3, wherein the fusion protein comprises presenilin 2 or a fragment thereof.

12. The method according to claim 1, wherein the method is a high throughput screening test.

13. The method according to claim 1, wherein the quantity of reporter cleaved is measured with luciferase, green fluorescent protein or a derivative thereof, or secretory alkaline phosphatase or β-galactosidase.

14. The method according to claim 1, wherein the quantity of reporter cleaved is detected indirectly.

15. The method according to claim 14, wherein a cleaved reporter domain migrates into the nucleus, binds there to another reporter domain of a reporter construct and the expression of the reporter is activated.

16. The method according to claim 1, wherein the cleaved reporter is Gal4.

17. The method according to claim 16, wherein the quantity of cleaved reporter Gal4 is detected indirectly, such that Gal4 in the nucleus binds to the Gal4DNA binding domain of another reporter construct and the expression of luciferase is activated.

18. The method according to claim 1, wherein the quantity of reporter cleaved is detected directly.

19. The method according to claim 1, wherein the cells are of neuronal or glial origin.

20. The method according to claim 1, wherein the cells are H4 cells.

21. A method for identifying substances capable of specifically inhibiting γ-secretase, comprising:
a) cultivating H4 cells which endogenously exhibit said γ-secretase activity and express the membrane-associated recombinant fusion protein Aβ-KKK/Gal4;
b) incubating said cells with a test substance;
c) measuring the quantity of transactivator Gal4 cleaved and binding to the Gal4-DNA binding domain of the luciferase construct by means of the level of luciferase expression; and
d) comparing the value obtained in step (c) with the value obtained in the absence of the test substance.

22. The method of claim 21, wherein the method is a high throughput screening test.

23. A substance identified by the method according to one of claims 1, 12, 21, or 22, wherein the substance specifically inhibits the proteolytic cleaving of a γ-secretase substrate.

24. A substance identified by the method according to one of claims 1, 12, 21, or 22, wherein the substance specifically inhibits the proteolytic cleaving of a presenilinase substrate.

25. A method for treating a neurodegenerative disorder comprising administering the substance according to claim 23.

26. A method for treating a neurodegenerative disorder comprising administering the substance according to claim 24.

27. The method according to claim 25, wherein the neurodegenerative disorder is Alzheimer's disease.

28. The method according to claim 26, wherein the neurodegenerative disorder is Alzheimer's disease.

29. A pharmaceutical composition comprising the substance according to claim 23; and a pharmaceutical carrier.

30. A pharmaceutical composition comprising the substance according to claim 24; and a pharmaceutical carrier.
